(19) United States
(12) Patent Application Publication
(10) Pub. No.: US 2014/0227326 A1
(43) Pub. Date: Aug. 14, 2014

(54) METHOD OF DELIVERING NASAL SPRAY

(71) Applicant: Novartis AG, Fort Worth, TX (US)

(72) Inventors: Gerald D. Cagle, Fort Worth, TX (US); G. Michael Wall, Fort Worth, TX (US)

(73) Assignee: Novartis AG, Fort Worth, TX (US)

(21) Appl. No.: 14/079,730

(22) Filed: Nov. 14, 2013

Related U.S. Application Data
(63) Continuation of application No. 11/263,240, filed on Oct. 31, 2005, now abandoned.
(60) Provisional application No. 60/630,886, filed on Nov. 24, 2004.

(51) Int. Cl. A61K 31/335; A61K 9/00

(52) U.S. Cl. CPC A61K 31/335; A61K 9/0043
USPC 424/400; 514/450

(57) ABSTRACT

A method of delivering a nasal spray. A sprayer having a formulation comprising olopatadine is provided. A spray of the formulation is delivered to a subject's nose. The spray may have a spray characteristic comprising a spray pattern having a longest axis of 20-45 mm, a shortest axis of 14-20 mm, and an ellipticity of 1-1.8. The spray may also have a spray characteristic comprising a droplet size distribution having a D₁₀ of 15-30 μm, a D₅₀ of 30-60 μm, a D₉₀ of 50-150 μm, a SPAN of not more than 3, and a % Volume of <10 μm of less than 4%.

### CROSS REFERENCE TO RELATED APPLICATION

This application is a continuation application of U.S. application Ser. No. 11/263240, filed Oct. 31, 2005, which claims priority of U.S. Provisional Application No. 60/630886 filed Nov. 24, 2004.

### FIELD OF THE INVENTION

The present invention generally pertains to the delivery of nasal sprays and more particularly to the delivery of nasal sprays containing olopatadine.

### DESCRIPTION OF THE RELATED ART

A variety of nasal sprays are available for treating allergic rhinitis. Exemplary products include FLONASE® nasal spray available from GlaxoSmithKline of the United Kingdom; NASONEX® nasal spray available from Schering Corporation of Kennilworth, N.J.; and ASTELIN® nasal spray available from MedPointe Pharmaceuticals of Somerset, New Jersey. All of these products deliver topical formulations via conventional pump-sprayers available from suppliers such as Pfeiffer of Germany; Saint-Gobain Calmar of France, or Valois of France.

U.S. Pat. Nos. 4,871,865 and 4,923,892, both assigned to Burroughs Wellcome Co. (“the Burroughs Wellcome Patents”), disclose that certain carboxylic acid derivatives of doxepin, including olopatadine (chemical name: Z-11-(3-dimethylaminopropylidene)-6,11-dihydrodibenz[b,e]oxepine-2-acetic acid), have antihistamine and antiasthmatic activity. The Burroughs Wellcome Patents teach various pharmaceutical formulations containing the carboxylic acid derivatives of doxepin, including nasal spray and ophthalmic formulations. See, for example, Col. 7, lines 7-26, and Examples 8 (H) and 8 (I) of the '865 patent.

U.S. Pat. No. 5,116,863, assigned to Kyowa Hakko Kogyo Co., Ltd., (“the Kyowa patent”), teaches that acetic acid derivatives of doxepin and, in particular, olopatadine, have anti-allergic and anti-inflammatory activity. Medicament forms taught by the Kyowa patent for the acetic acid derivatives of doxepin include a wide range of acceptable carriers; however, only oral and injection administration forms are mentioned.

U.S. Pat. No. 5,641,805, assigned to Alcon Laboratories, Inc. and Kyowa Hakko Kogyo Co., Ltd., teaches topical ophthalmic formulations containing olopatadine for treating allergic eye diseases. According to the '805 patent, the topical formulations may be solutions, suspensions or gels.

PATANOL® (olopatadine hydrochloride ophthalmic solution) 0.1%, from Alcon Laboratories, Inc. of Fort Worth, Tex., is currently the only commercially available olopatadine product for ophthalmic use. According to its labeling information, it contains olopatadine hydrochloride equivalent to 0.1% olopatadine, 0.01% benzalkonium chloride, and unspecified amounts of sodium chloride, dibasic sodium phosphate, hydrochloric acid and/or sodium hydroxide (to adjust pH) and purified water.

U.S. Patent Application Publication No. 20030055102 of Alcon, Inc. discloses topical olopatadine formulations that are effective for treating and/or preventing allergic or inflammatory disorders of the eye or nose. Formulations of aqueous solutions that comprise approximately 0.2-0.6% olopatadine are disclosed.

U.S. Pat. Nos. 4,871,865; 4,923,892; 5,116,863; and 5,641,805 and U.S. Patent Application Publication No. 20030055102 are incorporated herein in their entirety by this reference.

Improved methods of delivering topical olopatadine formulations that are effective for treating allergic or inflammatory conditions of the nose remain desirable.

### SUMMARY OF THE INVENTION

One aspect of the present invention is a method of delivering a nasal spray. A sprayer having a formulation comprising olopatadine is provided. A spray of the formulation is delivered to a subject's nose. The spray has a spray characteristic comprising a spray pattern having a longest axis of 20-45 mm, a shortest axis of 14-20 mm, and an ellipticity of 1-1.8.

In another aspect, the present invention is a method of delivering a nasal spray. A sprayer having a formulation comprising olopatadine is provided. A spray of the formulation is delivered to a subject's nose. The spray has a spray characteristic comprising a droplet size distribution having a D₁₀ of 15-30 μm, a D₅₀ of 30-60 μm, a D₉₀ of 50-150 μm, a SPAN of not more than 3, and a % Volume of <10 μm of less than 4%.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a front, sectional view of a nasal sprayer according to a preferred embodiment of the present invention.

### DETAILED DESCRIPTION OF THE PREFERRED EMBODIMENTS

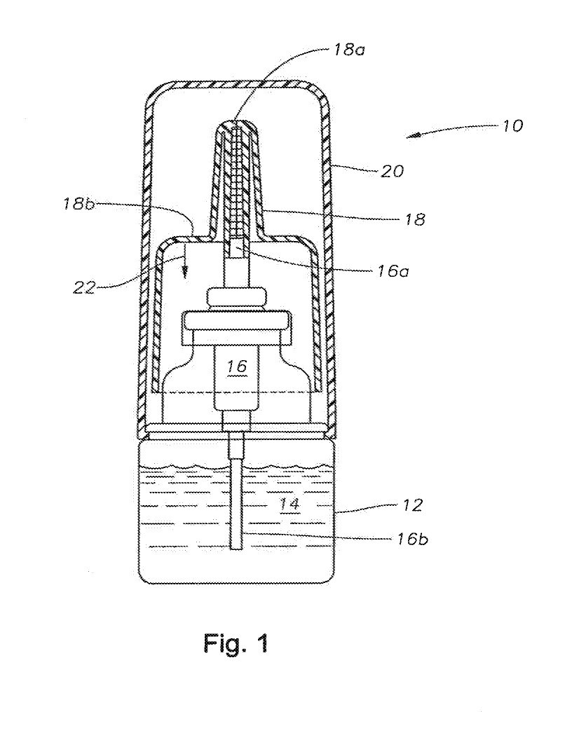

The preferred embodiments of the present invention and their advantages are best understood by referring to FIG. 1 of the drawings, like numerals being used for like and corresponding parts of the various drawings.

Unless indicated otherwise, all component amounts are presented on a % (w/v) basis and all references to olopatadine are to olopatadine free base.

FIG. 1 shows a nasal sprayer 10 according to a preferred embodiment of the present invention. Nasal sprayer 10 generally includes a bottle 12 holding a formulation 14, a pump 16 sealingly engaged with bottle 12, an actuator 18 removably receiving a top portion 16a of pump 16, and a cap 20 removably engaged with bottle 12 and for covering actuator 18. Bottle 12 and cap 20 are preferably made from high-density polyethylene. A preferred pump for pump 16 is the Valois VP7/100S CS20 pump. Actuator 18 is preferably made from polypropylene. Formulation 14 is preferably an aqueous formulation that is effective for treating and/or preventing allergic or inflammatory conditions in the nose containing olopatadine. Formulation 14 preferably contains 0.38-0.62% olopatadine. Formulation 14 most preferably contains 0.6% olopatadine. After removing cap 20 and inserting nozzle 18a of actuator 18 into his or her nose, a user may deliver a single spray of formulation 14 from bottle 12 via tube 16b of pump 16 and actuator 18 by moving surface 18b of actuator 18 in the direction of arrow 22. Nasal sprayer 10 may be manufactured using conventional techniques.

It has been discovered that for a formulation 14 containing 0.6% olopatadine and a viscosity of 1-2 cps (preferably 1.7 cps), a certain spray characteristic emitted from nozzle 18a results in unexpectedly beneficial clinical performance. More specifically, a spray characteristic having one or more of the following parameters is preferred: a shot weight of 90-110 mg, and most preferably 100 mg; a spray pattern having a longest axis of 20-45 mm, and most preferably 23.5 mm; a spray pattern having a shortest axis of 14-20 mm, and most preferably 17.5 mm; a spray pattern having an ellipticity of 1-1.8, more preferably 1-1.4, and most preferably 1.24; and the following droplet size distribution:
D₁₀=15-30 μm, more preferably 18-25 μm, and most preferably 22 μM;
D₅₀=30-60 μm, more preferably 39-53 μm, and most preferably 47 μm;
D90 =50-150 μm, more preferably 83-128 μm, and most preferably 106 μm;
SPAN =not more than 3, more preferably 1.6-2.1, and most preferably 1.8;
% Volume<10 μm=less than 4%, more preferably 0.8-2.4%, and most preferably 1.6%,
   where D₁₀ is the droplet size distribution of 10% of the droplets, D₅₀ is the droplet size distribution of 50% of the droplets, D₉₀ is the droplet size distribution of 90% of the droplets; SPAN is the ratio of (D₉₀-D₁₀)/D₅₀; and % Volume<10 μm is the percentage of droplets less than 10 μm in diameter. D₁₀, D₅₀, and D₉₀ are measurements of the diameter of droplets. When two sprays/nostril of a formulation 14 containing 0.6% olopatadine having all the preferred or more-preferred parameters of the above-referenced spray characteristic were delivered twice per day in clinical trials involving over 4000 human subjects, pharmacokinetic testing revealed that this method of delivery produced a particularly advantageous bioavailability of olopatadine. More specifically, a peak plasma concentration of olopatadine (C.) measured within 0.5-2 hours post-dose using high performance liquid chromatography from 14.4-35.3 ng/mL (mean 23.3 +/−6.1 ng/mL) was observed. This level of concentration is comparable to a concentration that would be expected to be obtained via a systemic (e.g. oral) dose form.

The following describes a preferred procedure for characterizing spray patterns. A TLC plate (e.g. silica gel 60, F₂₅₄ (florescence indicator), 250 μm thick layer on glass) and a TLC plate holder available from EM Science of Gibbstown, N.J.; a 254 nm filtered ultraviolet light source; and a camera suitable for taking pictures in ultraviolet light (e.g. a digital camera) are obtained. Sprayer 10 is loaded with formulation 14 and primed by actuating pump 16 via actuator 18 until a fine mist appears out of nozzle 18a. Sprayer 10 and the TLC plate holder are arranged so that nozzle 18a is about 3 cm from the TLC plate. Pump 16 is actuated via a conventional mechanical actuator using a constant force (preferably 5 kg). The resulting spray of formulation 14 is allowed to soak into the TLC plate. The TLC plate is moved to a dry section, and the procedure is repeated. For best results, two spray patterns are obtained from five separate units of sprayer 10. The patterns are viewed in 254 nm filtered ultraviolet light, and a photograph is taken of each pattern.

Using a printed photograph, each pattern is circled with a pencil. A single line is drawn to encircle all of the spray pattern, including any areas of density that appear to be apart from the rest of the pattern. The “outer ring”, which is sometimes visible and is the result of liquid spreading out on the plate after contact, should not be circled. The inner, darker, pattern is the original spray pattern to be measured. Using a pencil, the longest axis that can be found within each circled pattern is drawn. The shortest axis that passes through the center of each longest axis is drawn. Each axis is measured to the nearest 0.5 mm. The ellipticity (shape) of each pattern is calculated according to the following: ellipticity=longest axis/shortest axis. The ellipticity is reported to the nearest 1/10. The longest axis, shortest axis, and ellipticity of each pattern for each sprayer 10 are averaged to provide one set of parameters for each sprayer 10. The parameters for all five units of sprayer 10 are then averaged to find a single set of spray pattern parameters for a given formulation 14.

The following describes a preferred procedure for characterizing droplet size distribution. Sprayer 10 is loaded with formulation 14 and primed by actuating pump 16 via actuator 18 until a fine mist appears out of nozzle 18a. Sprayer 10 and a commercially available laser diffraction instrument are arranged so that nozzle 18a is about 5 cm below the laser beam of the laser diffraction instrument. Pump 16 is actuated via a conventional mechanical actuator using a constant force (preferably 5 kg). The resulting spray of formulation 14 crosses the laser beam. Data are collected for D₁₀, D₅₀, D₉₀, SPAN, and % Volume<10 gm. The average values for each of these parameters for two sprays are calculated.

From the above, it may be appreciated that the present invention provides improved methods of delivering topical olopatadine formulations that are effective for treating allergic or inflammatory conditions of the nose. It is believed that the operation and construction of the present invention will be apparent from the foregoing description. While the methods shown or described above have been characterized as being preferred, various changes and modifications may be made therein without departing from the spirit and scope of the invention as defined in the following claims.

What is claimed is:

1. A method of delivering a nasal spray comprising the steps of:
providing a sprayer having a formulation comprising olopatadine; and
delivering a spray of said formulation to a subject's nose having a spray characteristic comprising a spray pattern having a longest axis of 20-45 mm, a shortest axis of 14-20 mm, and an ellipticity of 1-1.8.

2. The method of claim 1 wherein said longest axis is 23.5 mm.

3. The method of claim 1 wherein said shortest axis is 17.5 mm.

4. The method of claim 1 wherein said ellipticity is 1-1.4.

5. The method of claim 1 wherein said formulation comprises 0.38-0.62% olopatadine.

6. The method of claim 5 wherein said formulation comprises 0.6% olopatadine.

7. The method of claim 1 wherein said spray characteristic further comprises a shot weight of 90-110 mg.

8. The method of claim 1 wherein said longest axis is 23.5 mm, said shortest axis is 17.5 mm, said ellipticity is 1-1.4, said formulation comprises 0.6% olopatadine, and said spray characteristic further comprises a shot weight of 90-110 mg.

9. A method of delivering a nasal spray comprising the steps of:
providing a sprayer having a formulation comprising olopatadine; and
delivering a spray of said formulation to a subject's nose having a spray characteristic comprising a droplet size distribution having a D₁₀ of 15-30 μm, a D₅₀ of 30-60 μm, a D₉₀ of 50-150 μm, a SPAN of not more than 3, and a % Volume of <10 lam of less than 4%.

10. The method of claim 9 wherein said formulation comprises 0.38-0.62% olopatadine.

11. The method of claim 10 wherein said formulation comprises 0.6% olopatadine.

12. The method of claim 9 wherein said spray characteristic further comprises a shot weight of 90-110 mg.

13. The method of claim 9 wherein D₁₀ is 18-25 μm, D₅₀ is 39-53 μm, D₉₀ is 83-128 μm, SPAN is 1.6-2.1, and % Volume of <10 μm is 0.8-2.4%.

14. The method of claim 9 wherein:
said droplet size distribution has a D₁₀ of 18-25 μm, a D₅₀ of 39-53 μm, a D₉₀ of 83-128 μm, a SPAN of 1.6-2.1, and a % Volume of <10 μm of 0.8-2.4%;
said formulation comprises 0.6% olopatadine; and
said spray characteristic further comprises a shot weight of 90-110 mg.

15. A method of delivering a nasal spray comprising the steps of:
providing a sprayer having a formulation comprising olopatadine; and
delivering a spray of said formulation to a subject's nose having a spray characteristic comprising:
a spray pattern having a longest axis of 20-45 mm, a shortest axis of 14-20 mm, and an ellipticity of 1-1.8; and
a droplet size distribution having a D₁₀ of 15-30 μm, a D₅₀ of 30-60 μm, a D₉₀ of 50-150 μm, a SPAN of not more than 3, and a % Volume of <10 μm of less than 4%.

16. The method of claim 15 wherein said formulation comprises 0.38-0.62% olopatadine.

17. The method of claim 15 wherein said formulation comprises 0.6% olopatadine.

18. The method of claim 15 wherein said spray characteristic further comprises a shot weight of 90-110 mg.

19. The method of claim 15 wherein said longest axis is 23.5 mm, said shortest axis is 17.5 mm, said ellipticity is 1-1.4, D₁₀ is 18-25 μm, D₅₀ is 39-53 μm, D₉₀ is 83-128 μm, SPAN is 1.6-2.1, % Volume of <10 μm is 0.8-2.4%, said formulation comprises 0.6% olopatadine, and said spray characteristic further comprises a shot weight of 90-110 mg.
